(19) United States
(12) Patent Application Publication
(10) Pub. No.: US 2025/0065317 A1
(43) Pub. Date: Feb. 27, 2025

(54) ENCAPSULATED CATALYTIC COMPOSITION

(71) Applicant: IFP ENERGIES NOUVELLES, Rueil-Malmaison (FR)

(72) Inventors: Christine DALMAZZONE, Rueil-Malmaison Cedex (FR); Cyril DARTIGUELONGUE, Rueil-Malmaison Cedex (FR); Severine FORGET, Rueil-Malmaison Cedex (FR); Lionel MAGNA, Rueil-Malmaison Cedex (FR); Minrui XU, Rueil-Malmaison Cedex (FR)

(73) Assignee: IFP ENERGIES NOUVELLES, Rueil-Malmaison (FR)

(21) Appl. No.: 18/722,050

(22) PCT Filed: Dec. 12, 2022
(86) PCT No.: PCT/EP2022/085455

§ 371 Date: Jun. 20, 2024

(30) Foreign Application Priority Data
Dec. 21, 2021 (EP) FR2114088

(51) Int. Cl. B01J 35/30; B01J 37/00; C07C 2/08

(52) U.S. Cl. CPC B01J 35/398; B01J 37/0072; C07C 2/08

(57) ABSTRACT

The present invention relates to a catalyst composition in the form of a capsule, having walls made of solid material which define a closed volume which contains a liquid phase comprising at least one ionic liquid of formula Q⁺A⁻, wherein Q⁺ is an organic cation and A⁻ is an anion, and in which a Brönsted acid HB is dissolved.

### TECHNICAL FIELD

The present invention relates to a catalyst composition and to its use in acid catalysis processes.

It relates more particularly to a composition resulting from the dissolution of at least one Brönsted acid, denoted HB, in an ionic liquid medium comprising at least one organic cation Q⁺ and one anion A⁻.

The present invention also relates to acid catalysis processes using said composition, and more particularly to alkylation of aromatic hydrocarbons, oligomerization of olefins, isomerization of n-olefins to iso-olefins, isomerization of n-paraffins to iso-paraffins, and alkylation of isobutane by olefins.

### PRIOR ART

Acid catalysis reactions are very important industrial reactions, which find very varied applications in the field of refining and petrochemicals. Reference may be made in particular to Christian Marcilly's book “Catalyse acido-basique—Application au raffinage et à la pétrochimie” [Acid-base catalysis—Application to refining and petrochemistry]—July 2003—Editions Technip, for more information on these reactions.

Mention may be made, by way of example, of the alkylation of aromatic hydrocarbons for the production of “linear alkyl benzenes”, LABs, which are intermediates for the synthesis of biodegradable detergents. Mention may also be made of the dimerization of isobutene to 2,4,4-trimethyl-1-pentene and 2,4,4-trimethyl-2-pentene which, after hydrogenation, leads to a desired additive for the reformulation of gasolines, 2,4,4-trimethylpentane (absence of sulfur, aromatic and olefin, high octane number, etc.).

The conventional acid catalysts used for these transformations are very often Lewis and/or Brönsted acids. The most commonly used are hydrofluoric acid (HF), concentrated sulfuric acid (H₂SO₄), boron trifluoride (BF₃) and aluminum trichloride (AlCl₃). However, the use of these acids has drawbacks, in particular because of the increasingly strict measures aimed at protecting the environment. For example, the use of HF, which is toxic, volatile and corrosive, involves the deployment of important safety measures for operators and equipment. Concentrated sulfuric acid, for its part, is not very active and requires the use of large volumes of acid, which generate discharges, essentially inorganic salts, which must be brought to environmental standards before being discharged. Aluminum trichloride, nevertheless still widely used industrially, pure or complexed with a base (often called “red oils”), is consumed in large quantities. However, this type of catalyst is not easily separated from the reaction products. From this point of view, solid catalysts such as zeolites or else sulfonic resins can provide an improvement as regards the separation of the products and the recycling of the catalyst, but they impose reaction temperatures which are often higher.

An alternative approach, for example described in patent FR 2 829 039, involves implementing this type of reactions and catalysts in the form of a two-phase liquid-liquid system: The Lewis acid and/or Brönsted acid is immobilized in an ionic liquid phase Q⁺A⁻, which is of low miscibility or immiscible with the reaction products. These products can then be separated by decantation, and the catalytic phase can then be recycled and reused. Catalyst consumption is thus reduced. However, these systems are not completely devoid of drawbacks: the viscosity of the ionic liquids entails, for example, the use of a mechanical energy that is high enough to ensure mixing between the two phases. The reaction selectivities can, moreover, be complicated to control, owing especially to follow-on reactions linked to the partial miscibility of the primary products in the acidic ionic liquid. (A follow-on reaction means a reaction where the reaction product reacts with the reagent to form a heavier, unwanted product)

Another approach involves implementing this type of reactions and catalysts in a continuous process, for example within a column-type reactor, in a fixed bed, with the immobilization of said catalyst composition in supported form. Continuous entrainment of the reaction products thus improves the selectivity and/or productivity of the process.

This approach makes it possible to combine the advantages of homogeneous catalysis (activity, selectivity) with the implementation in heterogeneous mode (recyclability of the catalyst, continuous operation, improved productivity).

Various approaches dedicated to the immobilization of ionic liquids in supported form are described in the prior art. Thus, the so-called SILP (acronym for the expression “supported ionic liquid phase”) technology involves impregnating a porous support with an ionic liquid, this technology being known as a means of heterogenizing an ionic liquid. Immobilization can be carried out by covalent bonds between the ionic liquid and the support, or else by physisorption via electrostatic interactions (Van der Waals, dipolar forces). Reference may be made to patent application US 2011/0318233A1 for the description of a reactor using an ionic liquid fixed on a porous solid support, for example silica gel, and a Brönsted acid.

The SILP technology nevertheless has drawbacks: the electrostatic bonds between the ionic liquid and the support are weak bonds, which can lead to a loss of ionic liquid by leaching over time. This type of implementation is therefore not very suitable for the immobilization of catalytic systems of interest to the present invention, for which recycling of the catalyst in its entirety, or at least in large part, is desired.

The object of the invention, then, is to improve the design of catalyst compositions operating in homogeneous liquid-liquid catalysis. The invention seeks in particular to improve their stability and/or their recyclability, or else to improve the yield and/or to improve the selectivity of the desired reactions.

### SUMMARY OF THE INVENTION

A first subject of the invention is a catalyst composition in the form of a capsule, having walls made of solid material which define a closed volume which contains a liquid phase comprising at least one ionic liquid of formula Q⁺A⁻, wherein Q⁺ is an organic cation and A⁻ is an anion, and in which a Brönsted acid HB is dissolved.

These capsules with a liquid core are very advantageous, and “work” as follows: they are brought into contact with the reactant or reactants in liquid form, for example in free suspension or in a fixed bed in a reactor. Their solid walls are chosen to be impervious with respect to the ionic liquid, but porous with respect to the reactants and the reaction products targeted: the reactants react by making contact with the ionic liquid of the capsules, and form reaction products which diffuse out of the capsules.

The invention has thus developed capsules with a liquid core, which have many advantages:

Firstly, these capsules stabilize the catalyst formulation; they immobilize the Q⁺A⁻/HB system, without releasing the Brönsted acid.

Next, these capsules are easily recyclable.

They are also easy to employ in reactors operating continuously, for example in a traversed bed, or else in a fluidized bed.

These capsules have a very good resistance to pressure (especially at least up to 10 bar), which makes it possible to use them in processes which require liquefaction of the reactant (for example in the case of an isobutene dimerization reaction).

These capsules allow highly viscous catalyst systems to be used in “heterogeneous” form, especially those based on formulations starting from ionic liquids. The solid capsules of the invention allow them to be employed more easily, without having to consider their high viscosity.

According to the invention, the concentration of Brönsted acid HB in the ionic liquid is preferably between 0.05 and 40% by weight, especially between 1 and 20% by weight.

The largest mean dimension of the capsules, especially their mean diameter in the case of substantially spherical capsules, is generally between 1 and 1000 μm, especially between 2 and 100 μm, or between 10 μm and 50 μm.

Advantageously, the organic cation Q⁺ may be a quaternary ammonium and/or a quaternary phosphonium and/or a trialkylsulfonium, and the anion A⁻ is an anion which with the cation Q⁺ forms a salt which is liquid below 150° C.

Advantageously, the anion A⁻ may be chosen from tetrafluoroborate, tetraalkylborate, hexafluorophosphate, hexafluoroantimonate, alkylsulfonate, especially methylsulfonate, perfluoroalkylsulfonate, especially trifluoromethylsulfonate, fluorosulfonate, sulfate, phosphate, perfluoroacetate, especially trifluoroacetate, perfluorosulfonamide, especially bis-trifluoromethanesulfonyl amide (CF₃SO₂)₂N⁻, fluorosulfonamide, perfluorosulfomethide, especially tris-trifluoromethanesulfonyl methide (CF₃SO₂)₃C⁻ and carboranes.

More preferably, the anion A⁻ is an anion which with the cation Q⁺ forms a salt which is liquid below 150° C.

The cation Q⁺ may especially be chosen from the following compounds:

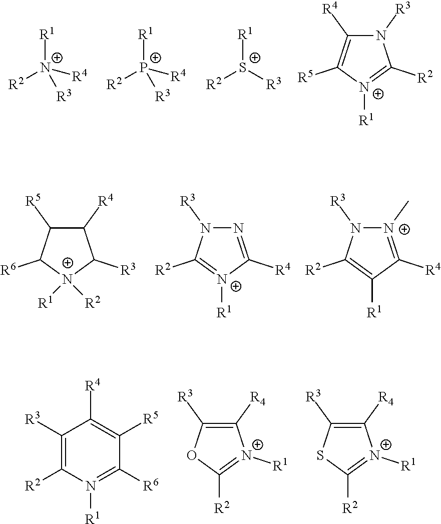

for which R¹, R², R³, R⁴, R⁵ and R⁶ are identical or different, bonded together or not, and represent hydrogen or hydrocarbyl groups having from 1 to 12 carbon atoms, especially alkyl groups, saturated or unsaturated, or cycloalkyl groups or aromatic aryl or aralkyl groups, comprising from 1 to 12 carbon atoms.

As examples of ionic liquids Q⁺A⁻ of interest according to the invention, mention may be made of N-butylpyridinium hexafluorophosphate, N-ethylpyridinium tetrafluoroborate, 3-butyl-1-methylimidazolium hexafluoroantimonate, 3-butyl-1-methylimidazolium hexafluorophosphate, 3-butyl-1-methylimidazolium trifluoromethylsulfonate, pyridinium fluorosulfonate, trimethylphenylammonium hexafluorophosphate, 3-butyl-1-methylimidazolium bis-trifluoromethylsulfonylamide, triethylsulfonium bis-trifluoromethylsulfonylamide, tributylhexylammonium bis-trifluoromethylsulfonylamide, 3-butyl-1-methylimidazolium trifluoroacetate, 3-butyl-1,2-dimethylimidazolium bis-trifluoromethylsulfonylamide. These salts can be used alone or as a mixture.

The Brönsted acids used according to the invention are defined as being acidic compounds capable of donating at least one proton. According to the invention, these Brönsted acids have the general formula HB, in which B represents an anion. Preferably, the Brönsted acid HB comprises an anion B chosen from the anions tetrafluoroborate, tetraalkylborates, hexafluorophosphate, hexafluoroantimonate, alkylsulfonates, especially methylsulfonate, perfluorosulfonate, especially trifluoromethylsulfonate, fluorosulfonate, sulfate, phosphate, perfluoroacetate, especially trifluoroacetate, perfluorosulfonamide, especially bis-trifluoromethanesulfonyl amide (CF₃SO₂)₂N⁻, fluorosulfonamide, perfluorosulfomethide, especially tris-trifluoromethanesulfonyl methide (CF₃SO₂)₃C⁻ and carborane.

The catalytic composition may comprise just one or several of these Brönsted acids, thus with different anions B. According to one embodiment, the Brönsted acid HB has the formula Q₂⁺A₂⁻, in which Q₂⁺ represents an organic cation comprising at least one sulfonic acid or carboxylic acid function, and A₂⁻ represents an anion, especially the same anion as the anion A⁻ of the ionic liquid.

Within the meaning of the invention, the term “sulfonic acid function” or “carboxylic acid function” means a hydrocarbyl substituent having from 1 to 12 carbon atoms, containing a sulfonic acid (—SO₃H) or carboxylic acid (—CO₂H) group grafted onto the cation Q₂⁺.

As examples of advantageous compositions Q₂⁺A₂⁻, mention may be made of 1-methyl-3-(2-ethylsulfonyl)imidazolium trifluoromethylsulfonate, 1-ethyl-3-(2-ethylcarboxyl)imidazolium bistriflylamide, N-butyl-N-(2-ethylsulfonyl)pyrrolidinium trifluoromethylsulfonate, N-ethyl-N-(2-ethylcarboxyl)pyrrolidinium bistriflylamide, (2-ethylsulfonyl)triethylammonium trifluoromethylsulfonate and triphenyl(3-propylsulfonyl)phosphonium para-toluenesulfonate.

The solid material of the walls of the capsule can be obtained from solid particles chosen from silica particles, preferably functionalized with hydrophobic hydrocarbon groups, clay particles, preferably modified with organic or amphiphilic molecules, magnetic nanoparticles, especially of Fe₃O₄, carbon nanotubes, particles of graphene oxides, particles of synthetic polymers, particles of a material of natural origin preferably chosen from hydroxyapatite, chitosan, cyclodextrin, dextran, particles in the form of cellulose nanocrystals or nanofibers, particles of biological material, especially of food grade, preferably chosen from starch, zein, soy proteins, bacteria and yeasts, with the optional addition to the particles of at least one surfactant. The optional surfactant, or each of the optional surfactants if there are several, may be of ionic, cationic or anionic, nonionic or amphoteric type.

The solid material of the walls of the capsule is obtained by said particles and by a crust formed by the addition of a crosslinking agent, which makes it possible to bond the solid particles together. According to one preferred embodiment, this crosslinking agent is chosen from at least one silicon compound of orthosilicate or alkoxysilane type, especially chosen from at least tetramethyl orthosilicate, tetraethyl orthosilicate, tetrabutyl orthosilicate, trimethoxysilane and triethoxysilane. This type of crosslinking agent is indeed capable of crosslinking by hydrolysis, and then serves as a base material for forming a solid crust.

According to one embodiment of the invention, the solid particles are chosen based on silicon oxide optionally functionalized with hydrophobic groups, and the crust is obtained by hydrolysis of a crosslinking agent chosen from the orthosilicates or alkoxysilanes mentioned above.

Another subject of the invention is a process for preparing the catalytic composition as described above.

This process may comprise the following steps:
(a) adding the Brönsted acid HB to the liquid comprising at least one organic cation Q⁺ and one anion A⁻ to obtain an ionic liquid denoted Q⁺A⁻/HB,
(b) adding the ionic liquid Q⁺A⁻/HB to a liquid phase L1 of hydrocarbon(s) containing solid particles, to form a so-called Pickering emulsion comprising droplets of the ionic liquid Q⁺A⁻/HB stabilized by the solid particles in the liquid phase L1 of hydrocarbon(s),
(c) adding a crosslinking agent to the emulsion obtained in step (b), in order to make it possible to bond together the solid particles to form solid walls surrounding the droplets of ionic liquid Q⁺A⁻/HB in the form of capsules of catalyst composition suspended in the liquid phase L1.

A further subject of the invention is the collective comprising the encapsulated catalytic compositions described above and the liquid phase L1 which was used for their preparation and in which the capsules are in suspension.

Pickering emulsions are liquid/liquid dispersions stabilized by nanoparticles or aggregates of solid nanoparticles which accumulate at the interface between the two immiscible liquids (generally water and oil) and prevent coalescence (see, for example, the publication Pickering, S. U. (1907). J. Chem. Soc. Trans. 91, 2001-2021). In fact, the particles used to make Pickering emulsions are capable of irreversibly attaching to the interface between the two liquids, causing much more effective stabilization of the emulsion than the adsorption of surfactants (see, for example, the publication Aveyard, R., Binks, B. P., and Clint, J. H. (2003). Adv. Colloid Interface Sci. 100, 503-546). The nature of the emulsion (water in oil or oil in water) is determined by the preferential wettability of the particles toward one or the other phase. In fact, the liquid which is the most wetting with respect to the particles will constitute the continuous phase of the emulsion, and the least wetting the dispersed phase (see, for example, the publication Binks, B., and Lumsdon, S. (2000). Langmuir 16, 8622-8631).

The capsules according to the invention are therefore prepared by passing through an intermediate step of forming such a so-called Pickering emulsion: instead of using as such the droplets of ionic liquid surrounded by solid particles, the invention creates a crust, a shell, a solid wall from these solid particles arranged around the droplets: encapsulated in this way, the droplets are more stable, easier to recycle and employ, and can also withstand higher pressure.

The ionic liquid Q⁺A⁻/HB may also comprise water, especially in a content of 0.1 to 10% by weight, preferably of 0.5 to 5% by weight and advantageously of 1 to 3% by weight relative to the ionic liquid Q⁺A⁻/HB.

The volume ratio between the ionic liquid Q⁺A⁻/HB and the liquid phase L1 of hydrocarbon(s) is preferably between 2:1 and 1:10, especially between 1:1 and 1:5.

In step (c), the crosslinking agent can be added by prior solubilization of said crosslinking agent in a liquid phase L2 of hydrocarbon(s), and it is therefore the liquid phase L2 containing the crosslinking agent that is added to the emulsion.

The solid particles may be chosen based on silicon compounds, especially silica, preferably functionalized with hydrophobic hydrocarbon groups.

The crosslinking agent may be chosen from at least one silicon compound of orthosilicate or alkoxysilane type, especially chosen from at least one of the following compounds: tetramethyl orthosilicate, tetraethyl orthosilicate, tetrabutyl orthosilicate, trimethoxysilane or triethoxysilane.

The presence of optional water in the ionic liquid can thus promote crosslinking of the crosslinking agent, when crosslinking takes place by hydrolysis.

Preferably, the mass ratio between the crosslinking agent and the solid particles is between 1 and 10, especially between 1 and 6.

The step (c) of crosslinking the solid particles is preferably carried out at a temperature of between 3° and 60° C., especially for 2 to 24 hours.

The preparation process according to the invention may also comprise a step (d) of separating the capsules of catalytic composition from the liquid phase L1 (or from at least part of said liquid phase).

The preparation process according to the invention may also comprise a step (e) of washing the capsules separated in step (d) one or more times, especially with a solvent L3 immiscible with the encapsulated catalyst composition, especially a non-aqueous solvent which preferably is based on hydrocarbon(s).

Preferably, in step (b) of formation of the Pickering emulsion, the amount of solid particles is between 0.1 and 10% by weight, preferably 0.5 to 5% by weight, especially 1 to 3% by weight, relative to the ionic liquid Q⁺A⁻/HB.

The solid particles which are suitable for the invention may be of various shapes and sizes (for example from a few nanometers to a few microns, in the form of substantially spherical beads or not). They may be of a single type, or they may be used as a mixture of several types of particles. They can be modified to change their surface properties (especially to modify their wettability).

The liquid phase L1 used in step (b) comprises one or more saturated hydrocarbons, especially of linear or cyclic alkane type, and/or one or more unsaturated hydrocarbons, in particular of olefin or aromatic compound type, said hydrocarbon or hydrocarbons preferably having between 3 and 20 carbon atoms, preferably between 5 and 9 carbon atoms.

Preferably, the first phase L1 comprises only one or more hydrocarbons. The first liquid phase L1 may be chosen from pentane, hexane, heptane, cyclohexane, methylcyclohexane, toluene and xylene, pure or as a mixture. Preferably, it is chosen from heptane, cyclohexane and methylcyclohexane. In the case where the first liquid phase L1 is an unsaturated hydrocarbon, it may be chosen from the products of the acid catalysis reaction implemented (alkylbenzene, di-isobutene, etc.).

The liquid phases L2 and L3, when used, may contain the same hydrocarbons as the liquid phase: either at least phase L2 and/or phase L3 have the same composition as phase L1, or they are at least chosen from the hydrocarbons or hydrocarbon mixtures mentioned above for phase L1.

Another subject of the invention is an acid catalysis process which uses the catalytic composition described above, in the form of capsules, or prepared according to the preparation process described above.

Another subject of the invention is an acid catalysis process wherein a catalytic composition in the form of a capsule is used, having walls made of solid material which define a closed volume which contains a liquid phase comprising at least one ionic liquid of formula Q⁺A⁻, wherein Q⁺ is an organic cation and A⁻ is an anion, and in which a Brönsted acid HB is dissolved.

The acid catalysis process can be carried out in a closed, semi-open or continuous system, with one or more reaction stages.

It may be a process of alkylation of aromatic hydrocarbons, oligomerization of olefins, dimerization of isobutene, isomerization of n-olefins to iso-olefins, isomerization of n-paraffins to iso-paraffins, and alkylation of isobutane by olefins.

It may thus be a process for alkylating aromatic hydrocarbons chosen from monocyclic aromatics, especially benzene, and alkylbenzenes such as toluene, ethylbenzene, xylene, mesitylene and durene, or polycyclic aromatics, especially naphthalene, alkylnaphthalenes and anthracene, said aromatic hydrocarbons being able to be substituted by one or more alkyl, aryl, alkylaryl, alkoxy, aryloxy or cycloalkyl groups, and/or by any group which does not interfere with the alkylation reaction, by at least one alkylating agent in the form of olefins having a number of carbon atoms of 2 to 20, these olefins being preferably chosen from ethylene, butenes, 1-hexene, 1-octene, 1-decene, 1-dodecene and 1-tetradecene, alone or as a mixture.

In this case, the molar ratio between the alkylating agent and the aromatic hydrocarbon is preferably between 0.05 to 100, and preferably between 0.1 and 10.

Advantageously, the temperature at which the aromatic alkylation is carried out may be between −50° C. and 200° C., being preferably less than 100° C. and especially between −20° C. and 50° C., in the presence or absence of vapor phase.

The reaction lasts preferably for between 1 minute and 10 hours.

The acid catalysis process may be an olefin isomerization process, especially for isomerizing at least one olefin having from 4 to 30 carbon atoms.

The acid catalysis process can be a process for dimerizing isobutene, starting from isobutene, pure or mixed with other hydrocarbons, optionally in the presence of an alcohol or an ether.

Preferably, the temperature at which the dimerization is carried out is between −50° C. to 200° C., being preferably less than 100° C.

The acid catalysis process can be carried out by reactive distillation.

The acid catalysis process can be a process for alkylating isoparaffin, especially isobutane, with olefins, said olefins being chosen from at least one of the following olefins: ethylene, butenes, 1-hexene, 1-octene, 1-decene, 1-dodecene, 1-tetradecene, alone or as a mixture.

In this case, the molar ratio between the isoparaffin and the olefin is preferably between 2/1 and 100/1, preferably between 10/1 and 50/1, especially between 5/1 and 20/1.

Advantageously, the temperature at which the alkylation of isoparaffin is carried out may be between −50 and 200° C., especially between −20 and 30° C.

### LIST OF FIGURES

FIG. 1 represents a scanning electron microscope image of capsules of catalyst composition prepared according to the invention.

For the purposes of the present invention, the various ranges of parameters for a given step, such as the pressure ranges and the temperature ranges, can be used alone or in combination.

For example, for the purposes of the present invention, a preferred range of pressure values can be combined with a range of more preferred temperature values.

In the text hereinbelow, the expressions “of between . . . and . . . ” and “between . . . and . . . ” are equivalent and mean that the limit values of the interval are included in the described range of values. Should such not be the case and should the limiting values not be included in the range described, such a clarification will be provided by the present invention.

### DESCRIPTION OF THE EMBODIMENTS

The invention relates to a catalyst composition in the form of a capsule, having walls made of solid material which define a closed volume which contains a liquid phase comprising at least one ionic liquid of formula Q⁺A⁻, wherein Q⁺ is an organic cation and A⁻ is an anion, and in which a Brönsted acid HB is dissolved, said catalyst composition being immobilized within capsules which comprise solid walls, also called “crust”, and a liquid core.

This crust is advantageously both porous with respect to the reactants and the products of the desired catalysis reaction, and impervious with respect to the ionic liquid.

(i)—The non-aqueous liquid medium in which the Brönsted acid HB according to the invention is dissolved has the general formula Q⁺A⁻ in which Q⁺ represents a quaternary ammonium and/or a quaternary phosphonium and/or a trialkylsulfonium (III) and A⁻ represents any known anion capable of forming, with the cation Q⁺, a salt which is liquid at low temperature, i.e. below 150° C.:

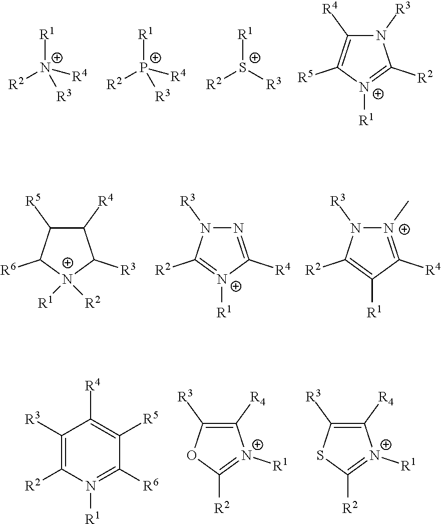

for which R¹, R², R³, R⁴, R⁵ and R⁶, which are identical or different, bonded together or not, represent hydrogen, hydrocarbyl radicals having from 1 to 12 carbon atoms, for example alkyl groups, saturated or unsaturated, or cycloalkyl groups or aromatic aryl or aralkyl groups, comprising from 1 to 12 carbon atoms,

the groups R¹, R², R³, R⁴, R⁵ and R⁶ include methyl, ethyl, propyl, isopropyl, butyl, secondary butyl, tertiary butyl, amyl, phenyl or benzyl radicals;

the ammonium and/or phosphonium cation is preferably chosen from the group formed by N-butylpyridinium, N-ethylpyridinium, 3-butyl-1-methylimidazolium, diethylpyrazolium, 3-ethyl-1-methylimidazolium, pyridinium, trimethylphenylammonium, tetrabutylphosphonium and methylethylpyrrolidinium.

The anions A⁻ which can be used in the context of the invention are preferably chosen from tetrafluoroborate anions, tetraalkylborates, hexafluorophosphate, hexafluoroantimonate, alkylsulfonates (for example methylsulfonate), perfluoroalkylsulfonates (for example trifluoromethylsulfonate), fluorosulfonate, sulfates, phosphates, perfluoroacetates (for example trifluoroacetate), perfluorosulfonamides (for example bis-trifluoromethanesulfonyl amide (CF₃SO₂)₂N⁻, fluorosulfonamides, perfluorosulfomethides (for example tris-trifluoromethanesulfonyl methide (CF₃SO₂)₃C⁻) and carboranes.

As examples of ionic liquids Q⁺A⁻ which can be used according to the invention, mention may be made of N-butylpyridinium hexafluorophosphate, N-ethylpyridinium tetrafluoroborate, 3-butyl-1-methylimidazolium hexafluoroantimonate, 3-butyl-1-methylimidazolium hexafluorophosphate, 3-butyl-1-methylimidazolium trifluoromethylsulfonate, pyridinium fluorosulfonate, trimethylphenylammonium hexafluorophosphate, 3-butyl-1-methylimidazolium bis-trifluoromethylsulfonylamide, triethylsulfonium bis-trifluoromethylsulfonylamide, tributylhexylammonium bis-trifluoromethylsulfonylamide, 3-butyl-1-methylimidazolium trifluoroacetate, 3-butyl-1,2-dimethylimidazolium bis-trifluoromethylsulfonylamide. These salts can be used alone or as a mixture.

(ii)—The Brönsted acids used according to the invention are defined as being acidic compounds capable of donating at least one proton. According to the invention, these Brönsted acids have the general formula HB, in which B represents an anion. The anions B are preferably chosen from tetrafluoroborate anions, tetraalkylborates, hexafluorophosphate, hexafluoroantimonate, alkylsulfonates (for example methylsulfonate), perfluorosulfonates (for example trifluoromethylsulfonate), fluorosulfonate, sulfates, phosphates, perfluoroacetates (for example trifluoroacetate), perfluorosulfonamides (for example bis-trifluoromethanesulfonyl amide (CF₃SO₂)₂N⁻, fluorosulfonamides, perfluorosulfomethides (for example tris-trifluoromethanesulfonyl methide (CF₃SO₂)₃C⁻) and carboranes. The Brönsted acids used according to the invention can be used alone or as a mixture.

According to the invention, the Brönsted acid HB may also have the general formula Q₂⁺A₂⁻, in which Q₂⁺ represents an organic cation comprising at least one sulfonic acid or carboxylic acid function, and A₂⁻ represents an anion (specify that these are the same anions as A⁻). The term “sulfonic acid or carboxylic acid function” is understood to mean a hydrocarbyl substituent having from 1 to 12 carbon atoms containing a sulfonic acid (—SO₃H) or carboxylic acid (—CO₂H) group grafted onto the cation Q₂⁺. Examples of the compositions Q₂⁺A₂⁻ which can be used include 1-methyl-3-(2-ethylsulfonyl)imidazolium trifluoromethylsulfonate, 1-ethyl-3-(2-ethylcarboxyl)imidazolium bistriflylamide, N-butyl-N-(2-ethylsulfonyl)pyrrolidinium trifluoromethylsulfonate, N-ethyl-N-(2-ethylcarboxyl)pyrrolidinium bistriflylamide, (2-ethylsulfonyl)triethylammonium trifluoromethylsulfonate and triphenyl(3-propylsulfonyl)phosphonium para-toluenesulfonate.

(iii)—The crust (or capsule wall) according to the invention consists of two elements:
on the one hand, the solid particles used in the preparation of the Pickering emulsion. These particles are advantageously silica particles having an affinity for each of the two phases. Given the hydrophobic nature of the hydrocarbon phase, the silica particles, which are naturally hydrophilic because of the presence of silanol groups, are preferably functionalized with hydrophobic hydrocarbon groups.

In addition to the modified silica particles, different types of organic and inorganic particles can be used to stabilize Pickering emulsions, as detailed in the review paper by Yang et al. (2017, Front. Pharmacol. 8:287). Mention will be made in particular of clays, which, like silica, can be easily modified with organic or amphiphilic molecules, but also in a non-exhaustive manner of magnetic nanoparticles (Fe₃O₄), carbon nanotubes, graphene oxides, synthetic polymers, natural products such as hydroxyapatite, chitosan, cyclodextrin, dextran, cellulose nanocrystals or nanofibers, and also food-grade biological particles such as starch, zein (corn protein), soy protein, wheat protein and even bacteria and yeasts. These particles are of various shapes and sizes (from a few nm to a few μm), can be used as such or as a mixture, and can be modified to change the surface properties (wettability).
on the other hand, a so-called crosslinking agent, which forms a porous crust, making it possible to bind the particles together.

According to one preferred variant, this agent crosslinks by hydrolysis, creating a crust that binds the particles together.

This agent may advantageously be a silicon compound of orthosilicate or alkoxysilane type. It may be, in a non-exhaustive manner, tetramethyl orthosilicate, tetraethyl orthosilicate, tetrabutyl orthosilicate, trimethoxysilane or triethoxysilane.

### Method of Preparing the Catalyst Composition

The invention also provides a method for preparing liquid-core capsules for encapsulating a Q⁺A⁻/HB catalyst formulation, comprising the following steps:
The Brönsted acid HB and water are added to the ionic liquid Q⁺A⁻ with magnetic stirring. The solid particles are dispersed in the hydrocarbon phase with magnetic stirring, or using a rotor-stator system like the system marketed under the name Ultra-Turrax.
The ionic liquid solution containing the Brönsted acid and water, denoted Q⁺A⁻/HB, is preferably added dropwise to the hydrocarbon phase (liquid phase L1) containing the solid particles with vigorous stirring, allowing the particles to take up position at the interface between the ionic liquid droplets and the hydrocarbon phase, and forming an ionic liquid-in-hydrocarbon Pickering emulsion. The dispersive energy is provided by any type of system providing energy to generate emulsification (rotor stator, propeller stirrer, static mixer, colloid mill, membrane system, ultrasonic agitation, microfluidic system, etc.).

The principle of these mixers is described for example in the Techniques de l'lngénieur file J2153V1: Procédés d'émulsification—Techniques d'appareillage [Emulsification processes—Equipment techniques], by M. Poux and JP Canselier, Oct. 6, 2004. A microfluidic system is described for example in the Techniques de l'lngénieur file J8010V1: Microfluidique et formulation—Emulsions et systèmes colloïdaux complexes [Microfluidics and formulation—Emulsions and complex colloidal systems], by V. Nardello-Rataj and J F Ontiveros, Oct. 5, 2019.
A solution containing the crosslinking agent solubilized in a hydrocarbon (liquid phase L2) is added to the Pickering emulsion obtained in the previous step. The collective is placed in a rotary system of the rotary evaporator type, and heated to the temperature enabling hydrolysis of the precursor to be carried out, for the appropriate period of time.

Preferably, the compositions of phases L1 and L2 are identical.

The upper hydrocarbon phase containing the residual precursor is then removed by withdrawal, and the liquid-core capsules thus obtained are washed several times with a hydrocarbon solvent immiscible with the encapsulated ionic liquid.

The amount of solid particles used to manufacture the Pickering emulsion is generally in the range from 0.1 to 10% by mass, preferably from 0.5 to 5% by mass, more preferably from 1 to 3% relative to the dispersed phase, namely the ionic liquid Q⁺A⁻/HB.

The amount of water introduced into the dispersed phase, namely the ionic liquid Q⁺A⁻/HB, lies in the range from 0.1 to 10% by mass, more preferably from 0.5% to 5% relative to the dispersed phase, namely the ionic liquid Q⁺A⁻/HB.

The volume ratio between the dispersed phase, namely the ionic liquid Q⁺A⁻/HB, and the hydrocarbon phase (phases L1+L2) is between 2:1 and 1:10, preferably between 1:1 and 1:5.

The concentration of Brönsted acid HB within the ionic liquid Q⁺A⁻ is generally between 0.05 and 40.0% by mass, preferably between 1 and 20%.

The stirring speed provided by the rotor-stator system for manufacturing the Pickering emulsion is generally between 1000 and 20 000 rpm, preferably between 2000 and 18 000 rpm, and very preferably between 5000 and 15 000 rpm.

The mass ratio between the siliceous precursor and the solid particles is generally between 1 and 10, preferably between 2 and 6.

The production of the crust of the capsules by hydrolysis of the precursor is carried out in a rotary system of the rotary evaporator type, at a temperature of between 30 and 60° C., for a heating period of between 2 and 24 h.

The preferred liquid-core capsules according to the invention have a number-average diameter of between 1 μm and 1000 μm, preferably between 2 μm and 100 μm.

The mean size of the capsules is measured by optical microscopy (Olympus BX51 with analySIS software for image analysis) or by scanning electron microscopy (SEM, ZEISS Supra 40 instrument).

### Applications in Acid Catalysis

According to the invention, the catalyst composition as defined above is used more particularly in acid catalysis processes, in particular in processes for alkylation of aromatic hydrocarbons, oligomerization of olefins, dimerization of isobutene, isomerization of n-olefins to iso-olefins, isomerization of n-paraffins to iso-paraffins, and alkylation of isobutane by olefins.

The reaction can be performed in a closed system, in a semi-open system or continuously with one or more reaction stages.

(i)—According to the invention, the acid catalysis process using the catalyst compositions defined above consists of an aromatic alkylation process.

The aromatic hydrocarbons considered according to the invention are monocyclic or polycyclic aromatics such as naphthalene, alkylnaphthalenes and anthracene. Monocyclic aromatics are benzene and alkylbenzenes (toluene, ethylbenzene, xylene, mesitylene, durene, etc.). These aromatics may be substituted by one or more alkyl, aryl, alkylaryl, alkoxy, aryloxy and cycloalkyl groups and/or by any group which does not interfere with the alkylation reaction.

The alkylating agents which can be used are olefins having a number of carbon atoms of 2 to 20. These olefins are more particularly ethylene, and also butenes, 1-hexene, 1-octene, 1-decene, 1-dodecene, 1-tetradecene, alone or as a mixture, as obtained, for example, in processes for production of alpha-olefins by oligomerization of ethylene or in processes for dehydrogenation of paraffins. These olefins can be used pure or diluted in an alkane.

The molar ratio between the olefin and the aromatic hydrocarbon can range from 0.05 to 100, and preferably from 0.1 to 10.

The temperature at which the aromatic alkylation is carried out ranges, for example, from −50° C. to 200° C.; it is advantageously less than 100° C. and preferably from −20° C. to 50° C. The reaction may take place in the presence or absence of a vapor phase and the pressure is the autogenous pressure;

The reaction time, which depends on the temperature, is between 1 minute and 10 hours. It is adjusted so as to find a good trade-off between conversion and selectivity.

(ii)—According to the invention, the acid catalysis process using the catalyst compositions defined above consists of a process for dimerizing isobutene.

The dimerization process according to the invention applies to isobutene pure or as a mixture with other hydrocarbons. The sources of isobutene are diverse. However, the most common are the dehydrogenation of isobutane and the dehydration of tert-butyl alcohol. The isobutene may also originate from a C4 cut from FCC (“fluid catalytic cracking”) or from steam cracking. In the latter case, isobutene can be used as a mixture with n-butenes, isobutane and butane. The process according to the invention then has the additional advantage of making it possible to selectively convert isobutene without having to separate it from the other constituents of the cut. Another advantage of the process according to the invention is that isobutene-butene co-dimerization can be limited. Isobutene can also come from a process for dehydrating biobased alcohol.

The dimerization reaction can be carried out in the presence of an alcohol or an ether.

The temperature at which the dimerization reaction is carried out ranges, for example, from −50° C. to 200° C.; it is advantageously less than 100° C.

The dimerization reaction can be performed using a reactive distillation technique.

(iii)—According to the invention, the acid catalysis process using the catalyst compositions defined above consists of a process for alkylating isobutane by olefins.

The olefins which can be used in the isobutane alkylation process are chosen more particularly from ethylene, butenes, 1-hexene, 1-octene, 1-decene, 1-dodecene and 1-tetradecene, alone or as a mixture, as obtained, for example, in processes for production of alpha-olefins by oligomerization of ethylene or in processes for dehydrogenation of paraffins. These olefins can be used pure or as a mixture.

In the present process, the iso-paraffin and the olefin may be introduced separately or as a mixture. The molar ratio between the iso-paraffin and the olefin is, for example, from 2/1 to 100/1 and more advantageously from 10/1 to 50/1, preferably from 5/1 to 20/1.

The temperature at which the aliphatic alkylation reaction is carried out ranges, for example, from −50° C. to 200° C.; preferably, it is from −20° C. to +30° C.

(iv)—According to the invention, the acid catalysis process using the catalyst compositions defined above consists of an olefin isomerization process.

The process for isomerizing the double bond of olefins described according to the invention applies to olefins having from 4 to 30 carbon atoms, pure or as a mixture.

The temperature at which the isomerization reaction is carried out ranges, for example, from −50° C. to 200° C.; it is advantageously less than 100° C.

The isomerization reaction can be performed using a reactive distillation technique.

The invention will be described in greater detail with the aid of the non-limiting examples below.

### EXAMPLES

### Example 1 (Comparative): Pressure Resistance of a Pickering Emulsion in a Fixed Bed

5.6 g of 1-butyl-3-methylimidazolium bis(trifluoromethanesulfonyl)imide ([BMI][NTf₂]), 0.014 g of HNTf₂ acid and a few milligrams of 4-nitroaniline, a dye which has a yellow color in this mixture, are introduced into a beaker, under an inert atmosphere. This mixture is denoted phase 1.

5.13 g of n-heptane and then 0.145 g of silica particles (Aerosil R972, sold by Evonik) are introduced into another beaker, under an inert atmosphere. These particles are dispersed in the n-heptane with magnetic stirring. This mixture is denoted phase 2.

The phase 1 mixture is poured dropwise into the beaker containing n-heptane and silica (phase 2), with continuous application of a dispersion energy supplied by a rotor-stator system (UltraTurrax, 10 000 rpm).

A [BMI][NTf₂]/HNTf₂/SiO₂/heptane Pickering emulsion is thus obtained.

A piece of glass wool and then a layer of fine sand (2.5 g) are introduced into the bottom of the tubular reactor described in example 4. All of the Pickering emulsion described in this example is placed above the bed of sand. The upper volume of the reactor is then made up with heptane.

The reactor is then closed and connected at the inlet to a ballast containing n-heptane, maintained at a pressure of 10 bar. The valve is opened and the liquid feed is introduced into the reactor. The pressure is adjusted by means of a pressure reducer placed upstream of the reactor, and a pressure regulator placed at the outlet of the reactor. The pressure is gradually increased.

As soon as the pressure reaches approximately 2 bar, the effluent collected is yellow in color, and the dispersed phase of the emulsion containing the dye therefore leaves the column reactor. The Pickering emulsion is broken. Consequently, the Pickering emulsion thus used in a column reactor does not withstand a pressure of a few bars.

### Example 2 (According to the Invention): Preparation of Capsules Containing the Catalyst System [BMI][NTf₂]/[BMI][BF₄]/HNTf₂

16.9 g of 1-butyl-3-methylimidazolium bis(trifluoromethanesulfonyl)imide ([BMI][NTf₂]), 4.5 g of 1-butyl-3-methylimidazolium tetrafluoroborate [BMI][BF₄], 3.0 g of HNTf₂ acid and 0.6 g of water are introduced into a beaker, under an inert atmosphere. A few milligrams of 4-nitroaniline are added to this mixture, a dye which has a yellow color in this mixture. This mixture is denoted phase 1.

In another beaker, 0.422 g of silica fume (Aerosil R972, sold by Evonik) are dispersed in 27.3 mL (18.7 g) of n-heptane with magnetic stirring. This mixture is denoted phase 2.

The phase 1 mixture is poured dropwise into the beaker containing n-heptane and silica (phase 2), with continuous application of a dispersion energy supplied by a rotor-stator system (UltraTurrax, 10 000 rpm).

The Pickering emulsion thus obtained is transferred into a flask of greater capacity. A solution of tetramethyl orthosilicate (TMOS) at 7 g/L (1.74 g of TMOS in 250 mL of heptane) is then poured gently into the flask to trigger the sol-gel process. The flask is processed on a rotary evaporator in an oil bath at 50° C. for 12 hours.

After decantation, the upper phase containing the heptane and the residual TMOS is withdrawn by cannula and then eliminated. The capsules obtained are then washed 3 times with an n-heptane solution.

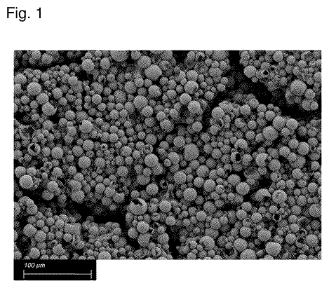

FIG. 1 is an image of the capsules thus prepared, obtained by scanning electron microscopy (SEM, ZEISS Supra 40 instrument), by observation with secondary electrons. It can be seen that the capsules are more in the form of substantially spherical beads, having a mean diameter as measured by SEM of 15 μm.

### Example 3 (According to the Invention): Pressure Resistance of Liquid-Core Capsules in a Fixed Bed

A piece of glass wool and then a layer of fine sand (2.5 g) are introduced into the bottom of the tubular reactor described in example 4. An amount of 13 g of capsules synthesized according to example 2 is taken up with a little heptane and placed above the bed of sand. The upper volume of the reactor is then made up with heptane.

The reactor is then closed and connected at the inlet to a ballast containing n-heptane, maintained at a pressure of 10 bar. The valve is opened and the liquid feed is introduced into the reactor. The pressure is adjusted by means of a pressure reducer placed upstream of the reactor, and a pressure regulator placed at the outlet of the reactor. The pressure is gradually increased to approximately 8 bar. The effluent collected is transparent (absence of dye), which reflects the fact that the catalyst composition thus used remains well confined inside the column reactor, within the capsules. The capsules therefore withstand a pressure of approximately 8 bar.

### Example 4 (According to the Invention): Dimerization of Isobutene in a Fixed Bed Using the Capsules Described in Example 2

A tubular stainless steel reactor with a length of 10 cm and an internal diameter of 1 cm is used for the employment of the capsules in isobutene dimerization.

A piece of glass wool and then a layer of fine sand (2.5 g) are introduced into the bottom of the tubular reactor. An amount of 13.3 g of capsules synthesized according to example 1 is taken up with a little heptane and placed above the bed of sand. The upper volume of the reactor is then made up with heptane.

The reactor is then closed and connected at the inlet to a ballast containing a mixture of 15% by mass of isobutene, 5% by mass of n-butane and 80% by mass of n-heptane, maintained at a pressure of 10 bar. The valve is opened, and the liquid feed is introduced into the reactor. The pressure is adjusted by means of a pressure reducer placed upstream of the reactor, and a pressure regulator placed at the outlet of the reactor. A pressure of 4 bar is thus applied. The outlet valve located downstream of the pressure regulator is opened to continuously collect the effluents leaving the reactor.

The effluents withdrawn are then analyzed by gas chromatography (PONA column) after treatment with sodium hydroxide (0.1 M) in order to eliminate possible traces of acid and drying over MgSO₄.

Isobutene conversion is measured using butane present in the feed as internal standard. (As a reminder, the role of the internal standard is as follows: The internal standard is a compound inert with respect to the chemical reaction carried out, which is introduced in a known amount into the reaction medium. It makes it possible to measure the amount of products formed after reaction, by gas chromatography, as follows (schematically: the chromatographic area of the internal standard peak, which corresponds to the mass which was initially introduced, is measured, then the area of the products is measured, and by rule of three the mass of these products is deduced therefrom).)

The selectivity for isobutene dimers (olefins containing 8 carbon atoms) corresponds to the mass ratio between the dimers and all of the products formed (C₈, C₁₂, C₁₆).

After 1 hour, the liquid flow rate at the reactor outlet stabilizes at around 50 mL/hour.

The sampling carried out after 3 hours shows a mass conversion of isobutene of 96%. The selectivity for dimerization products (C₈ olefins) and trimerization products (C₁₂) is 69% and 31%, respectively.

The sampling carried out after 5 hours shows a mass conversion of isobutene of 95%. The selectivity for dimerization products (C₈ olefins) and trimerization products (C₁₂) is 66% and 34%, respectively. After 5 hours, the effluent is still transparent.

1. A catalyst composition in the form of a capsule, having walls made of solid material which define a closed volume which contains a liquid phase comprising at least one ionic liquid of formula Q⁺A⁻, wherein Q⁺ is an organic cation and A⁻ is an anion, and in which a Brönsted acid HB is dissolved.

2. The catalyst composition as claimed in claim 1, wherein the concentration of Brönsted acid HB in the ionic liquid is between 0.05 and 40% by weight.

3. The catalyst composition as claimed in claim 1, wherein the organic cation Q⁺ is a quaternary ammonium and/or a quaternary phosphonium and/or a trialkylsulfonium, and in that the anion A⁻ is an anion which with the cation Q⁺ forms a salt which is liquid below 150° C.

4. The catalyst composition as claimed in claim 1, wherein the anion A⁻ is chosen from tetrafluoroborate, tetraalkylborate, hexafluorophosphate, hexafluoroantimonate, alkylsulfonate, especially methylsulfonate, perfluoroalkylsulfonate, especially trifluoromethylsulfonate, fluorosulfonate, sulfate, phosphate, perfluoroacetate, especially trifluoroacetate, perfluorosulfonamide, especially bis-trifluoromethanesulfonyl amide (CF₃SO₂)₂N^{×}, fluorosulfonamide, perfluorosulfomethide, especially tris-trifluoromethanesulfonyl methide (CF₃SO₂)₃C⁻, and carboranes.

5. The catalyst composition as claimed in claim 1, wherein the cation Q⁺ is chosen from the following compounds 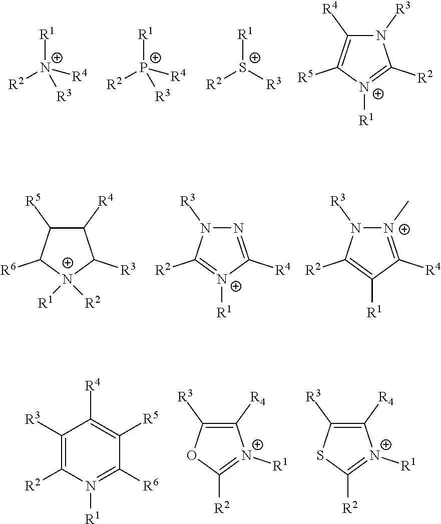
for which R¹, R², R³, R⁴, R⁵ and R⁶ are identical or different, bonded together or not, and represent hydrogen or hydrocarbyl groups having from 1 to 12 carbon atoms, especially alkyl groups, saturated or unsaturated, or cycloalkyl groups or aromatic aryl or aralkyl groups, comprising from 1 to 12 carbon atoms.

6. The catalyst composition as claimed in claim 1, wherein the Brönsted acid HB comprises an anion B chosen from the anions tetrafluoroborate, tetraalkylborates, hexafluorophosphate, hexafluoroantimonate, alkylsulfonates, especially methylsulfonate, perfluorosulfonate, especially trifluoromethylsulfonate, fluorosulfonate, sulfate, phosphate, perfluoroacetate, especially trifluoroacetate, perfluorosulfonamide, especially bis-trifluoromethanesulfonyl amide (CF₃SO₂)₂N⁻, fluorosulfonamide, perfluorosulfomethide, especially tris-trifluoromethanesulfonyl methide (CF₃SO₂)₃C⁻ and carborane.

7. The catalyst composition as claimed in claim 1, wherein the Brönsted acid HB has the formula Q₂⁺A₂⁻, in which Q₂⁺ represents an organic cation comprising at least one sulfonic acid or carboxylic acid function, and A₂⁻ represents an anion, especially the same anion as the anion A⁻ of the ionic liquid.

8. The catalyst composition as claimed in claim 1, wherein the solid material of the walls of the capsule is obtained from solid particles chosen from silica particles, preferably functionalized with hydrophobic hydrocarbon groups, clay particles, preferably modified with organic or amphiphilic molecules, magnetic nanoparticles, especially of Fe₃O₄, carbon nanotubes, particles of graphene oxides, particles of synthetic polymers, particles of a material of natural origin preferably chosen from hydroxyapatite, chitosan, cyclodextrin, dextran, particles in the form of cellulose nanocrystals or nanofibers, particles of biological material, especially of food grade, preferably chosen from starch, zein, soy proteins, bacteria and yeasts, with the optional addition to the particles of at least one surfactant.

9. The catalyst composition as claimed claim 8, wherein the solid material of the walls of the capsule is obtained by said particles bonded together by at least one crosslinking agent, chosen especially from at least one silicon compound of orthosilicate or alkoxysilane type, especially chosen from at least tetramethyl orthosilicate, tetraethyl orthosilicate, tetrabutyl orthosilicate, trimethoxysilane and triethoxysilane.

10. A process for preparing the catalyst composition as claim 1, comprising:
(a) adding the Brönsted acid HB to the liquid comprising at least one organic cation Q⁺ and one anion A⁻ to obtain an ionic liquid denoted Q⁺A⁻/HB,
(b) adding the ionic liquid Q⁺A⁻/HB to a liquid phase L1 of hydrocarbon(s) containing solid particles, to form a so-called Pickering emulsion comprising droplets of the ionic liquid Q⁺A⁻/HB stabilized by the solid particles in the liquid phase L1 of hydrocarbon(s), and
(c) adding a crosslinking agent to the emulsion obtained in (b), in order to form a crust making it possible to bond together the solid particles to form solid walls surrounding the droplets of ionic liquid Q⁺A⁻/HB in the form of capsules of catalyst composition suspended in the liquid phase L1.

11. The process as claimed in claim 10, wherein the ionic liquid Q⁺A⁻/HB also comprises water, especially in a content of 0.1 to 10% by weight, preferably of 0.5 to 5% by weight and advantageously of 1 to 3% by weight relative to the ionic liquid Q⁺A⁻/HB.

12. The process as claimed in claim 10, wherein the solid particles are based on silicon compounds, especially silica, preferably functionalized with hydrophobic hydrocarbon groups, and in that the crosslinking agent is chosen from at least one silicon compound of orthosilicate or alkoxysilane type, especially chosen from at least one of the following compounds: tetramethyl orthosilicate, tetraethyl orthosilicate, tetrabutyl orthosilicate, trimethoxysilane or triethoxysilane.

13. The process as claimed in claim 10, wherein the mass ratio of the crosslinking agent to the solid particles is between 1 and 10, especially between 1 and 6.

14. The process as claimed in claim 10, further comprising (d) separating the capsules of catalyst composition from the liquid phase L1.

15. The process as claimed in claim 14, further comprising (e) washing the capsules separated in step (d) one or more times, especially with a solvent L3 immiscible with the encapsulated catalyst composition, especially a non-aqueous solvent which preferably is based on hydrocarbon(s).

16. The process as claimed in claim 10, wherein in (b), formation of the Pickering emulsion, the amount of solid particles is between 0.1 and 10% by weight, preferably 0.5 to 5% by weight, especially 1 to 3% by weight, relative to the ionic liquid Q⁺A⁻/HB.

17. An acid catalysis process, especially chosen from processes of alkylation of aromatic hydrocarbons, oligomerization of olefins, dimerization of isobutene, isomerization of n-olefins to iso-olefins, isomerization of n-paraffins to iso-paraffins, and alkylation of isobutane by olefins, characterized in that said process uses a catalyst composition in the form of a capsule having walls of solid material which define a closed volume which contains a liquid phase comprising at least one ionic liquid of formula Q⁺A⁻, Q⁺ being an organic cation and A⁻ being an anion, and in which a Brönsted acid HB is dissolved.

18. The catalyst composition as claimed in claim 1, wherein the concentration of Brönsted acid HB in the ionic liquid is between 0 1 and 20% by weight.

19. The catalyst composition as claimed in claim 1, wherein the anion A⁻ is chosen from tetrafluoroborate, tetraalkylborate, hexafluorophosphate, hexafluoroantimonate, methylsulfonate, trifluoromethylsulfonate, fluorosulfonate, sulfate, phosphate, trifluoroacetate, bis-trifluoromethanesulfonyl amide (CF₃SO₂)₂N⁻, fluorosulfonamide, tris-trifluoromethanesulfonyl methide (CF₃SO₂)₃C⁻, and carboranes.

20. The catalyst composition as claimed in claim 5, wherein R¹, R², R³, R⁴, R⁵ and R⁶ are identical or different, bonded together or not, and represent hydrogen, or alkyl groups, saturated or unsaturated, cycloalkyl groups, aromatic aryl, aralkyl groups, in each case having up to 12 carbon atoms.
